SEQUENCE LISTING (46 sequences)

SEQ ID NO: 1 - protein, 251 aa, Danio rerio
MADGENKRAESAEPDEPLPKKPRLLELSGDSEHSATAGADTLDEKPARMDESQQALSINNNNNTRPTEPGQPADPEPEISELTDEGVHPNGFTSPDLLRDDDDCSSRASSSDWTPQPQIGSYRFIQQHIMRGTDPRAILKDLLPETVLPPDLDDMTLWQIIINISEPPKRKKRKDINTLEDVVRLLNERKKILVLTGAGVSVSCGIPDFRSRDGIYARLAVDFPDLPDPQAMFDIDYFRRDPRPFFKFAKV

SEQ ID NO: 2 - protein, 379 aa, Danio rerio
MSEEVSKRVEEEADTPGLEGQSDDSSDEGDASGDTEMDFLRSLFSRTLGLSPGDKVLDELTLDSVARYILSGKCKNIICMVGAGISTSAGIPDFHSPGTGLYANLQKYNLPYPEAIFQIDYFKKHPEPFFALARELYPGQFKPTVCHYFIKMLKDKGLLRRCYSQNIDTLERVAGLEGEDLIEAHGTFHTSHCVSFLCRKEYSMDWMKNQIFSEEIPKCDSCGSLVKPDIVFFGESLPSRFFTSMKADFPQCDLLIIMGTSLQVQPFASLVSRVSNRCPRLLINMEKTGQSEFGMGLFSFGGGMDFDSDKAYRDVAHLSTCDDGCMTLAELLGWKKELEEMVKREHALIDSKDAKKTDKEASQSSKSAVAEAEKTDKTE

SEQ ID NO: 3 - protein, 357 aa, Danio rerio
MLYLNTFLPSVCRRCFAENLLWRRGLTTTQNLSRTKLVHQKTLSHFPHAQKGAAFLSQFIYCPAAFIKCGGTRGLFGGGRDNVHQQTLEDIAEKIRERKFKRIVVMAGAGISTPSGIPDFRSPGSGLYDNLQQYNLPYAEAIFEINYFHHNPNPFFALAKELYPGNYQPNLTHYFIRMLHDKEQLLRMYTQNIDGLERMAGIPPKMLVEAHGTFATATCTVCRRDYKGEELRDDIMAGTVPKCPTCKGIIKPDIVFFGEELPQHFFTYLTDFPIADLLIVMGTSLEVEPFASLAGAVRGSVPRLLINRDLVGPFASGSQRHTDVAELGDVVNGVKKLVELLGWKQELEDLMNVGRDK

SEQ ID NO: 4 - protein, 370 aa, Danio rerio
MSKARLSRDRRAASVGVSRVTRSSMMSPQDCERSRAPDPGLLDELSLMSVSEQQASATRKGSSKPALSSPSGRSVSRGALETIGRLMKLGRVRNIVVVAGAGISTASGIPDFRTPGTGLYANLAKYDIPYPEAVFNIDYFSDNPHPFFSLAKELYPGHHRPNYVHYFIRMLHQKGLLLRMYTQNIDGLEKLCGIPDDKLVEAHGSFATAACHLCYTPYPAEEAKQAIMNGSVPICTFCAGAVKPNVVFFGEDLPEKYFQHAEDFPKADLLIIMGTSLKIEPFASLINTVKSTVPRLLLNRDAVGPFERRPLRRADYMELGDLSESVRKLAEILGWHTEIQTLMNSHENGLYSYISSSGENSGDSETDSMH

SEQ ID NO: 5 - protein, 310 aa, Danio rerio
MLLSCRYLPPPVAVGRCASTIQAGVRQFVPASGSFDSSALEQLQAFISQASRLFVISGAGLSTESGIPDYRSEGVGLYARTNRRPMQHSEFVRSEKSRQRYWARNYVGWPQFSSHQPNSAHLALRDWEEKGKLHWLVTQNVDALHLKAGQQRLTELHGSTHRVVCLDCGELTPRAELQKRFTALNPGWEATACAVAPDGDVFLEEEQVLNFRVPACNACGGVLKPEVTFFGDVVNRNTVHFVHNKLAESDAVLVAGSSLQVFSGYRFLLAASERKLPIAIVNIGATRADHLTDIRVSARCGEVLPAIKLS

SEQ ID NO: 6 - protein, 305 aa, Danio rerio
MIVRQLWCSRGSTSHLCAAVRLNWRSPKMTRPSSDLTAFREHFAKAKHIAIITGAGVSAESGVPTFRGPGGFWRKWQAQDLATPEAFSRDPSLVWEFYHYRREVMRSKMPNPAHLAIAECEARLGQQGRSVVIITQNIDELHHRAGSKHVYEIHGSLFKTRCMSCGEVKANHKSPICPALDGKGAPDPNTKEARIPVELLPRCERKSCNGLLRPHVVWFGETLDSDILTAVERELEKCDLCLVVGTSSIVYPAAMFAPQVASRGVPVAEFNMECTPATQRFKYHFEGPCGSTLPPALERHESEAV

SEQ ID NO: 7 - protein, 354 aa, Danio rerio
MSVNYAAGLSPYADKGICGLPETFDSPEELKTKVETLAQWIRESQYMVVHSGAGISTSTGIPDFRGPNGVWTMEERGETPHFNTTFEDARPSLTHMALLQMQRTGHLKYLISQNVDGLHVRSGFPRDRLSELHGNMFVEECEKCGKQYVRDTVVGVMGLKPTGRYCDVMRSRGLRSCRGKLISSILDWEDSLPDRDLNRADEASRRADLALTLGTSLQIKPSGDLPLLTKRTGGKLVIVNLQPTKHDKHAHLRIYGYVDDVMGQLMKLLGLDVPEWAGPTLCEDSGGDLDILPYGAWKKEVKIELKIEESNHTVSKKRKRKEQHAEEGYKNGVKVEEEMKEEGKESDSHVHTHT

SEQ ID NO: 8 - protein, 397 aa, Danio rerio
MDVRINSGVSARAERKEQEKAKIIQREKQRQTMKTISKILQKCESEWTEEERSMLQAHQDTVQELSRRQNRRHLLKRKQEEMFDDAENLKTKVKQLAEAVQRAKHLVIYTGAGISTAASIPDYRGPNGVWTQLQKGRSVSTSDLSQAEPTLTHMSIWMLHKMKMVRNCTSFMWTPLHVTLSSPVSCVSQVCDSCSPPREFIRLFDVTERTALHRHGTGRSCPHCRAELRDTIVHFGERGTLEQPLNWKGAAEAAQRADLILCLGSSLKVLKKYSCLWCMNRPASKRPKLYIVNLQWTPKDNLATLKIHGKCDAVMALLMEELALAGPVYSRLQDPIFSMAKPLSPQEQKSHSRKEIAPPSALEEVSQSAPPQGEGPAVQGGWFGRGYSKGRRKKKSS

SEQ ID NO: 9 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 10 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 11 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 12 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 13 - protein, 310 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 14 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 15 - protein, 400 aa, Homo sapiens
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 16 - protein, 756 aa, Gallus gallus
MADGEAPLLRPRDGGPGAAAESVEPAPKRQRLNSEDGVCGRGAPPAHRPDRGAGPPPAAAAATEPPGDAAAVSADGDVRAREEDGGATTEGRSGADNRAAQRGLARAEPPPQPRRQGRGEGAEAAPGEDAAEAAIGCERAQRSNGAAGAPAPQPDNFLLSDEIIANGFHSCDSDEDDRASHASSSDWTPRPRIGPYTFVQQHLMLGTDPRTILKDLLPETIPPPELDDMTLWQIVINILSEPPKRKKRKDVNTIDDAVKLLQECKKIMVLTGAGVSVSCGIPDFRSRDGIYARLAVDFPDLPDPQAMFDIEYFRKDPRPFFKFAKEIYPGQFQPSLCHKFIALMDKEGKLLRNYTQNIDTLEQVAGIQRIIQCHGSFATASCLICKYKVDCEVVRGDIFNQVVPRCPRCLPDEPLAIMKPDIVFFGENLPEQFHRAMKYDKNEVDLLIVIGSSLKVRPVALIPSSIPHEVPQILINREPLPHLHFDVELLGDCDVIISELCQRLGSEYTKLCYNSVKLSEITEKPPRMHKELEMHSSELPPTPLDISEDSGSPEQMTPPGTSVVPSEHAAECKVENSDPASETKGICTEEKLQDTQASSENPENPASELMNSETMKENGTNNGESKEKNEIVKKCWVNRSAKEQISKRLDGTQYLFLPPNRYIFHGAEVYSDSEDDMISSSSCGSSSESGSCHSQSLDVEDESEIEEFYNGIEDEDAPEREVEAAFEEDGVEQDAADESAYTNEAAGNDHPTSNKL

SEQ ID NO: 17 - protein, 388 aa, Gallus gallus
MSEADAPPPGADPDAEAEGGSEEDSDMELLRNLLARTLGLGTEPPERVLDELSLAGIARFMQSERCRRVVCMVGAGISTAAGIPDFRSPGTGLYANLGRYELPYPEAIFDISYFKQHPEPFFALAKELLPGQLKPTVCHYFMRLLKEKGLLLRCYTQNIDTLERVAGLQPEELVEAHGTFQTAHCLRSSCRHQYDLSWVKEKIFSSLVPKCDKCQSVVKPDIVFFGENLPSRFFSLLQSDFQKVDLLIIMGTSLQVQPFASLVSRVPASTPRLLINKEKTGQSDVFMSLMGFGCGMDFDSDKAYRDVAWLGDCDEGCLALAELLGWKKELQELVRKEHAAIDAVAAPEDTSSASGGDPTSRRGRTDGSGGRAESGGASSEQRGDDKEP

SEQ ID NO: 18 - protein, 346 aa, Gallus gallus
MERGVRRGAALVAAWRSLWERGGLALFRPQCRTGCGACRVQGTRPFSLSAAASAVLGLGSWGGDSGKQKLTLQDVAELIRKKECRRVVVMAGAGISTPSGIPDFRSPGSGLYSNLEQYNIPYPEAIFELAYFFINPKPFFTLAKELYPGNYRPNYAHYFLRLLHDKGLLLRLYTQNIDGLERVAGIPPDRLVEAHGTFATATCTVCRRKFPGEDFRGDVMADKVPHCRVCTGIVKPDIVFFGEELPQRFFLHMTDFPMADLLFVIGTSLEVEPFASLAGAVRNSVPRVLINRDLVGPFAWQQRYNDIAQLGDVVTGVEKMVELLDWNEEMQTLIQKEKEKLDAKDK

SEQ ID NO: 19 - protein, 512 aa, Gallus gallus
MAGYWEKFSTSNNRASDKPHWLRYCHCAKLTAPSAPPTRPEAAPPPTSQRAGALPRAPHLRPRAPPAAAAPRSERRCVRGGARRSGAAAELERGVCREPAMVMSAGRAAPAREEGLRRAAEGGCEALGAVPGLYPCRKLGFCSLNHGNCSRCAELCTDCISHKALLAGSRSRSWQRYERAFRVCAKANAVCGPRSLNMFPARRLPGGCRAIRPHHLRHHSVPSASPNLAFVPACLPPHPAEVEEMQRFISNSKKLFVMTGAGISTESGIPDYRSEGVGLYARSDRRPIQHAEFVRSATARQRYWARNFVGWPQFSSHQPNTAHLVLRHWEKLGKLHWLVTQNVDALHTKAGSQRMTELHGCTHRVFCLTCGDQTSRSELQEHFEALNPGWKAEALGVAPDGDAFLTDEQVRNFQVPACRKCGGILKPDVTFFGDTVSREKVDFVHQRLAESDSMLVAGSSMQVYSGYRFALAAREKQLPIAVLNIGPTRLDHFASLKLNSRCGELLPLIVAH

SEQ ID NO: 20 - protein, 423 aa, Gallus gallus
MGDVPRSEAGPRPAVPAAAFGCSAALRGSLQPGSAAAFSEEPLRTAPQAAAPPPAAARRAKKQPRRGGGAPRCRGAEGGPGRAGDVRRRTAGLSGAEERPALRLPSGQRIVAVLMCLAQCAARRLLAQVHGALKPAPKKPNWCWEMARPSSNMADFREVFAKAKHIAIITGAGVSAESGVPTFRGAGGFWRKWQAQELATPGAFARNPSRVWEFYHYRREVMLSKHPNAAHIAIAECEKRLRKQGRSVVVITQNIDELHRKAGTKHLLEIHGSLFKTRCTNCGNVTANYKSPICPALAGKGAPDPEIEDAAIPVEELPQCEEDGCHGLLRPHVVWFGETLDPDVLTEVEKELDLCDLCLVVGTSSVVYPAAMFAPQVSARGVPVAEFNMEATPATDRFRFHFEGPCGTTLPPALARHESEIIA

SEQ ID NO: 21 - protein, 357 aa, Gallus gallus
MAVNYAAGLSPYSDKGKCGLPEIFDPPEELERKVCELADLIRSSSNVVFHTGAGISTASGIPDFRGPNGVWTMEEKGLSPKFDTTFENARPSKTHMALLGLQRVGILKFLVSQNVDGLHVRSGFPRDKLAELHGNMFVEECMKCGKQYVRDAVVGSMGLKPTGRLCSVTKARGLRACRGKLRDTILDWEDSLPDRDLTLADEACRKADLSVTLGTSLQIKPSGNLPLITKKRGGKLVIVNLQATKHDRQADLRIHAYVDDVMTKLLKHLGLEVPEWTGPVVVESADSAKPEQLYTFKPEAHGLLKEEPFSQHNGTAGQCPDLGTTLVEHRDSLKQECPSPDTGPPLTKKMKVEPLLT

SEQ ID NO: 22 - protein, 399 aa, Gallus gallus
MAAGGSLSRSERKAAARAEILQQEEQRDRRRQVSRIWRKPPAERSPEECQVLSESEDIVRELERRRKRRERLRRRQEEVCDEPEELKRKVVELAAAIRNAKHLVIYTGAGISTAASIPDYRGPNGIWTLLQKGRSISTTDLSEAEPTLTHMSIACLHKHNLVQHVVSQNCDGLHLRSGLPRSAISELHGNMYIEVCTSCTPNREYVRVFDVTERTALHKHHTGRMCHKCGAQLRDTIVHFGEKGTLTQPLNWEAATEAASKADVILCLGSSLKVLKKYPRLWCMSKPPPRRPKLYIVNLQWTPKDDLAALKLHGRCDDVMRLLMEELGLQIPGYERARDPIFALAEPLRPEEEGTHSRKPVAPPQGTEPAAAPRDEPPARPGGWLGRGCAKGTRRRKSN

SEQ ID NO: 23 - protein, 737 aa, Mus musculus
MADEVALALQAAGSPSAAAAMEAASQPADEPLRKRPRRDGPGLGRSPGEPSAAVAPAAAGCEAASAAAPAALWREAAGAAASAEREAPATAVAGDGDNGSGLRREPRAADDFDDDEGEEEDEAAAAAAAAAIGYRDNLLLTDGLLTNGFHSCESDDDDRTSHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRILQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRPQKELVHLSELPPTPLHISEDSSSPERTVPQDSSVIATLVDQATNNNVNDLEVSESSCVEEKPQEVQTSRNVENINVENPDFKAVGSSTADKNERTSVAETVRKCWPNRLAKEQISKRLEGNQYLFVPPNRYIFHGAEVYSDSEDDVLSSSSCGSNSDSGTCQSPSLEEPLEDESEIEEFYNGLEDDTERPECAGGSGFGADGGDQEVVNEAIATRQELTDVNYPSDKS

SEQ ID NO: 24 - protein, 389 aa, Mus musculus
MAEPDPSDPLETQAGKVQEAQDSDSDTEGGATGGEAEMDFLRNLFTQTLGLGSQKERLLDELTLEGVTRYMQSERCRKVICLVGAGISTSAGIPDFRSPSTGLYANLEKYHLPYPEAIFEISYFKKHPEPFFALAKELYPGQFKPTICHYFIRLLKEKGLLLRCYTQNIDTLERVAGLEPQDLVEAHGTFYTSHCVNTSCRKEYTMGWMKEKIFSEATPRCEQCQSVVKPDIVFFGENLPSRFFSCMQSDFSKVDLLIIMGTSLQVQPFASLISKAPLATPRLLINKEKTGQTDPFLGMMMGLGGGMDFDSKKAYRDVAWLGDCDQGCLALADLLGWKKELEDLVRREHANIDAQSGSQAPNPSTTISPGKSPPPAKEAARTKEKEEQQ

SEQ ID NO: 25 - protein, 257 aa, Mus musculus
MVGAGISTPSGIPDFRSPGSGLYSNLQQYDIPYPEAIFELGFFFHNPKPFFMLAKELYPGHYRPNVTHYFLRLLHDKELLLRLYTQNIDGLERASGIPASKLVEAHGTFVTATCTVCRRSFPGEDIWADVMADRVPRCPVCTGVVKPDIVFFGEQLPARFLLHMADFALADLLLILGTSLEVEPFASLSEAVQKSVPRLLINRDLVGPFVLSPRRKDVVQLGDVVHGVERLVDLLGWTQELLDLMQRERGKLDGQDR

SEQ ID NO: 26 - protein, 333 aa, Mus musculus
MSGLTFRPTKGRWITHLSRPRSCGPSGLFVPPSPPLDPEKIKELQRFISLSKKLLVMTGAGISTESSIPDYRSEKVGLYARTDRRPIQHIDFVRSAPVRQRYWARNFVGWPQFSSHQPNPAHWALSNWERLGKLHWLVTQNVDALHSKAGSQRLTELHGCMHRVLCLNCGEQTARRVLQERFQALNPSWSAEAQGVAPDGDVFLTEEQVRSFQVPCCDRCGGPLKPDVVFFGDTVNPDKVDFVHRRVKEADSLLVVGSSLQVYSGYRFILTAREQKLPIAILNIGPTRSDDLACLKLDSRCGELLPLIDPRRQHSDVQRLEMNFPLSSAAQDP

SEQ ID NO: 27 - protein, 310 aa, Mus musculus
MRPLLIAPGRFISQLCCRRKPPASPQSKICLTMARPSSNMADFRKCFANAKHIAIISGAGVSAESGVPTFRGAGGYWRKWQAQDLATPQAFARNPSQVWEFYHYRREVMRSKEPNPGHLAIAQCEARLRDQGRRVVVITQNIDELHRKAGTKNLLEIHGTLFKTRCTSCGTVAENYRSPICPALAGKGAPEPETQDARIPVDKLPRCEEAGCGGLLRPHVVWFGENLDPAILEEVDRELALCDLCLVVGTSSVVYPAAMFAPQVASRGVPVAEFNMETTPATDRFRFHFPGPCGKTLPEALAPHETERTS

SEQ ID NO: 28 - protein, 334 aa, Mus musculus
MSVNYAAGLSPYADKGKCGLPEIFDPPEELERKVWELARLMWQSSSVVFHTGAGISTASGIPDFRGPHGVWTMEERGLAPKFDTTFENARPSKTHMALVQLERMGFLSFLVSQNVDGLHVRSGFPRDKLAELHGNMFVEECPKCKTQYVRDTVVGTMGLKATGRLCTVAKTRGLRACRGELRDTILDWEDSLPDRDLMLADEASRTADLSVTLGTSLQIRPSGNLPLATKRRGGRLVIVNLQPTKHDRQADLRIHGYVDEVMCRLMKHLGLEIPAWDGPCVLDKALPPLPRPVALKAEPPVHLNGAVHVSYKSKPNSPILHRPPKRVKTEAAPS

SEQ ID NO: 29 - protein, 402 aa, Mus musculus
MAAGGGLSRSERKAAERVRRLREEQQRERLRQVSRILRKAAAERSAEEGRLLAESEDLVTELQGRSRRREGLKRRQEEVCDDPEELRRKVRELARAVRSARHLVVYTGAGISTAASIPDYRGPNGVWTLLQKGRPVSAADLSEAEPTLTHMSITRLHEQKLVQHVVSQNCDGLHLRSGLPRTAISELHGNMYIEVCTSCIPNREYVRVFDVTERTALHRHLTGRTCHKCGTQLRDTIVHFGERGTLGQPLNWEAATEAASKADTILCLGSSLKVLKKYPRLWCMTKPPSRRPKLYIVNLQWTPKDDWAALKLHGKCDDVIQLLMNELGLEIPVYSRWQDPIFSLATPLRAGEEGSHSRKSLCRSREEAPPGDQSDPLASAPPILGGWFGRGCAKRAKRKKVA

SEQ ID NO: 30 - DNA, 1852 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 31 - DNA, 25 bp, Artificial Sequence
  Description of Artificial Sequence Synthetic primer
cagctctgctacaattcatcgcgtc

SEQ ID NO: 32 - DNA, 26 bp, Artificial Sequence
  Description of Artificial Sequence Synthetic primer
aatctctgtagagtccagcgcgtgtg

SEQ ID NO: 33 - DNA, 29 bp, Artificial Sequence
  Description of Artificial Sequence Synthetic primer
tctctgaagaaattcctaagtgcgattcc

SEQ ID NO: 34 - DNA, 28 bp, Artificial Sequence
  Description of Artificial Sequence Synthetic primer
ttatctgaatcaaaatccattccgcctc

SEQ ID NO: 35 - DNA, 28 bp, Artificial Sequence
  Description of Artificial Sequence Synthetic primer
cattaaatgtggtggaacaagaggcctg

SEQ ID NO: 36 - DNA, 28 bp, Artificial Sequence
  Description of Artificial Sequence Synthetic primer
agttcctctcctttgtaatccctccgac

SEQ ID NO: 37 - DNA, 24 bp, Artificial Sequence
  Description of Artificial Sequence Synthetic primer
cggcaggctgatgaagcttggtcg

SEQ ID NO: 38 - DNA, 25 bp, Artificial Sequence
  Description of Artificial Sequence Synthetic primer
tagcttgcttggcttcctctgcagg

SEQ ID NO: 39 - DNA, 28 bp, Artificial Sequence
  Description of Artificial Sequence Synthetic primer
tgtggtgaactgactcctcgtgctgagc

SEQ ID NO: 40 - DNA, 28 bp, Artificial Sequence
  Description of Artificial Sequence Synthetic primer
cggaagttttctttcactagcagcgagg

SEQ ID NO: 41 - DNA, 27 bp, Artificial Sequence
  Description of Artificial Sequence Synthetic primer
ccacggtagtctgtttaaaacccgctg

SEQ ID NO: 42 - DNA, 28 bp, Artificial Sequence
  Description of Artificial Sequence Synthetic primer
agtgatatttgaagcgttgggtagcagg

SEQ ID NO: 43 - DNA, 25 bp, Artificial Sequence
  Description of Artificial Sequence Synthetic primer
ggactgggaggactctctgcccgac

SEQ ID NO: 44 - DNA, 20 bp, Artificial Sequence
  Description of Artificial Sequence Synthetic primer
gcccggcccactccggaacg

SEQ ID NO: 45 - DNA, 26 bp, Artificial Sequence
  Description of Artificial Sequence Synthetic primer
gcattttggagaacgtggcactttgg

SEQ ID NO: 46 - DNA, 26 bp, Artificial Sequence
  Description of Artificial Sequence Synthetic primer
gtttagccatgctgaagatggggtcc

(19) United States
(12) Patent Application Publication
(10) Pub. No.: US 2010/0184869 A1
(43) Pub. Date: Jul. 22, 2010

(54) METHOD AND KIT FOR DETERMINING SIRTUIN MODULATING AGENTS, SIRTUIN MODULATING PROCEDURE, SIRTUIN MODULATING COMPOUNDS AND COMPOSITIONS INCLUDING THE SAME

(76) Inventors: Mauricio Reis Bogo, (US); Andre Arigony Souto, (US); Carla Denise Bonan, (US); Talita Cameiro Brandao Pereira, (US); Helena Schirmer, (US); Eduardo Pacheco Rico, (US); Denis Broock Rosemberg, (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/578,876

(22) Filed: Oct. 14, 2009

(30) Foreign Application Priority Data
Oct. 17, 2008 (BR) PI0806044-4

(51) Int. Cl. A61K 31/05; C12Q 1/68; C12N 5/02; C12N 5/07

(52) U.S. Cl. 514/733; 435/6; 435/375; 435/349; 435/354; 435/366

(57) ABSTRACT

A kit and a method for determining modulating agents of sirtuins; specifically, the modulation of sirtuin expression, by way of the detection and comparison of mRNAs in the sirtuins and β-actin. A process for modulating the activity of sirtuins, as well as pharmaceutical compounds and compositions capable of modulating the gene expression of sirtuins.

### STATEMENT OF RELATED APPLICATIONS

This patent application is based on and claims convention priority on Brazilian Patent Application No. PI0806044-4 having a filing date of 17 Oct. 2008.

### BACKGROUND OF THE INVENTION

1. Technical Field

The invention herein describes a kit and a method for determining sirtuin modulating agents; specifically the modulation of the expression of sirtuins, by way of the detection and comparison of mRNAs of sirtuins and of β-actin.

The invention herein also provides a process for the modulation of sirtuin activity as well as pharmaceutical compounds and compositions capable of modulating the gene expression of sirtuins.

2. Related Art

Sirtuins are part of a large group of enzymes, the histone deacetylases or HDACs family, whose principal role is to reverse the regulatory acetylation of histone-type proteins, by influencing directly in the structure of nucleosomes and, consequently, in gene transcription. Other than histones, a growing number of proteins have been identified as a target of HDACs, structural proteins and different transcription factors being among those. Sirtuins affect a broad number of physiological processes, including regulation of life expectancy, regulation of metabolic and enzymatic activity, cellular response to stress, neurodegeneration, DNA repair, rDNA recombination, apoptosis and the control of cellular proliferation. Currently, sirtuins are the important targets of research linked to caloric restriction, cancer, neurodegenerative diseases, muscular differentiation, inflammation and obesity.

Different studies have been performed aiding in the identification of sirtuin modulators. Blander et al (“SIRT1 shows no substrate specificity in vitro” J Biol Chem. 280(11):9780-5, 2005) describe a previously unpublished method for identifying specificity for a sequence of deacetylase by using peptide libraries containing acetylated lysine. After incubation with SIRT1, the subassembly of deacetylated peptides was captured selectively using a biotinylated N-hydroxysuccinimide photolabile linker and small beads of streptavidin, subsequently analysed. Said studies revealed that the recognition of the substrate by SIRT1 occurs irrespectively of the sequence of amino acids close to the acetylated lysine. Furthermore, Garske et al. (“SIRTI top 40 hits: use of one-bead, one-compound peptide libraries and quantum dots to probe deacetylase specificity.” Biochemistry 45(I):94-101, 2006) also describe an new method of high scalability for determining the specificity of deacetylase substrates using an acetylated peptide library of one-bead, one-compound (OBOC). A library of 104,907 unique peptides was constructed and screened utilising NAD+− dependent deacetylase SIRT1 for more efficient peptide sequences. As a result, it was discovered that SIRT1 can discriminate between peptide substrates depending on the context in which they find themselves.

The document, WO 08/27379, describes a method for monitoring the modulation of sirtuin activity by determining the level of acetylation of sirtuin substrates.

Documents, WO 07/149270 and WO 06/096780, describe a method for screening modulating molecules of sirtuin activity, which include as a crucial stage the determination of the level of acetylation of a specific peptide.

The document, WO 07/084162, describes new molecules with sirtuin inhibiting activity that are useful in the treatment and/or prevention of cancer and autoimmune diseases.

The abovementioned studies illustrate the difficulties and discrepancies that arise from methods for identifying specific substrates for sirtuins. With regard to peptide libraries, such libraries were limited to small sequences and are influenced by amino acids that can be incorporated and they provide information about artificial sequences in an artificial context.

As the methods proposed by publicly disclosed research do not demonstrate a consensus regarding their results, one can see that there exists a need for a method for identifying sirtuin modulators and in an appropriate cellular environment. The identification of modulating agents will help to clarify the role of sirtuins in cells and will aid in the development of diagnostic tests and the treatment of patients with modulators of the respective activity, as well as methods for identifying new modulating compounds.

The invention herein is different from all the documents relating to publicly disclosed research, as it proposes a new method for determining the modulation of sirtuin activity through the modulation of its gene expression. None of the documents from publicly disclosed research describes or even suggests that sirtuin activity can be modulated with agents that modulates its expression. Until now, publicly disclosed research has been concerned solely with the modulation of enzymatic sirtuin activity, which has already been expressed in the cell.

### BRIEF SUMMARY OF THE INVENTION

Firstly, the invention herein provides a method and a kit for determining modulating agents of sirtuin gene expression.

An object of this invention is therefore a method for determining modulating agents comprised of the following stages:
a) contacting tissue cell(s) comprising at least of gene of the sirtuin family and the β-actin gene, with a modulating agent;
b) determining the relative abundance of mRNA of gene(s) of the sirtuin family;
c) determining the relative abundance of mRNA of the β-actin gene; and
d) determining the mRNA SIRTImRNA β-actin ratio.

In particular, tissue cell(s) comprising the abovementioned genes are those of zebrafish.

A further object of the invention herein is a kit for determining modulating agents comprising:
a) means for determining the relative abundance of mRNA of sirtuin-family genes; and
b) means for determining the relative abundance of mRNA of the β-actin gene.

Secondly, the invention herein provides a process for modulating sirtuins, in which the modulation of sirtuins is performed by way of the modulation of the gene expression of sirtuins.

An additional object of the invention herein is a modulating agent of sirtuin expression.

Another object of the invention herein is a pharmaceutical composition comprising:
a) a modulating agent of sirtuin expression; and
b) an acceptable pharmaceutical vehicle.

### BRIEF DESCRIPTION OF THE DRAWING FIGURES

Further features of the invention can be gathered from the accompanying drawings, in which:

FIG. 1 demonstrates one of the trees generated that was constructed utilising a proportional distance (p-distance) by way of the Neighbour-Joining (NJ) method, using the MEGA4.02 program. The tree contains seven well-settled terminal cladistic groupings with a high support amount, corresponding to each one of the sirtuins described. The phylogenetic tree consistently grouped together the orthological sequences, SIRT1-SIRT7 of Danio rerio (Dr), Gallus gallus (Gg), Mus musculus (Mm) and Homo sapiens (Hs)

FIG. 2 demonstrates the pattern of expression of SIRT1 (A), SIRT2 (B), SIRT3 (C), SIRT3.2 (D), SIRT4 (E), SIRT5 (F), SIRT6 (G) and SIRT7 (H) in zebrafish. These results are expressed as an mRNA ratio of sirtuin/β-actin (mean ±S.E.) from seven repeated, independent experiments. On the Y axis, I is the rate of mRNA, on the X axis, 1 is the spleen, 2 is the gills, 3 is the brain, 4 is the heart, 5 is the liver, 6 is the female sexual organ, 7 is the male sexual organ, 8 is muscle and 9 is the kidney, I is β-actin, II is SIRT1, Ill is SIRT2, IV is SIRT3, V is SIRT3.2, VI is SIRT4, VII is SIRT5, VIII is SIRT6, IX is SIRT7.

FIG. 3 demonstrates the evaluation of the effect of resveratrol in the modulation of sirtuin activity in zebrafish in different tissues in 4 animals (groups 1 to 4 on the X axis). On the Y axis, I is the rate of mRNA X, a is muscle and b is liver.

### DETAILED DESCRIPTION OF PREFERRED EMBODIMENTS

The examples shown here have the single aim of exemplifying one of the numerous ways of carrying out the invention, however, without detracting from its purpose.

Sirtuins

For the purpose of this invention, the expression, “sirtuins”, refers to the sequences (DNA and/or RNA), as well as the respective proteins, comprising at least 50% of homology with the sequences (DNA and/or RNA), as well as the respective proteins of the chosen genes of the group comprising SIRT1, SIRT2, SIRT3, SIRT4, SIRT5, SIRT5, SIRT6, SIRT7 and combinations of the same.

The respective sequences of the SIRT1, SIRT2, SIRT3, SIRT4, SIRT5, SIRT6 and SIRT7 genes are described in the GenBank, under the access codes for the species, Danio rerio (XP_{—}001334440, AAH67165, XP_{—}684225, XP_{—}690925 (SIRT 3.2), AAH83418, AAH75987, NP_{—}001002071, XP_{—}698800), Homo sapiens (Q96EB6, Q8IXJ6, Q9NTG7, Q9Y6E7, Q9NXA8, Q8N6T7, Q9NRC8), Mus musculus (BAD38898, BAS38897, XP_{—}420920, XP_{—}415273, XP_{—}418925, NP_{—}001034409, NP_{—}001026277) and Gallus gallus (NP_{—}062786, NP_{—}071877, CAJ18608, Q8R216, NP_{—}849179, NP_{—}853617, AAP83960).

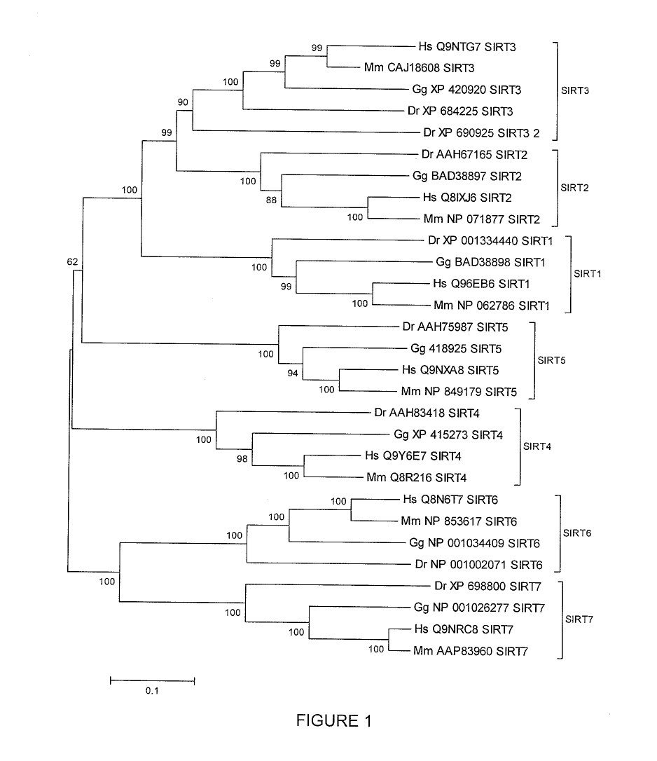

Ideally, sirtuins that are useful in the invention herein possess DNA sequences with at least 50% homology with the sequences illustrated in FIG. 1.

β-Actin

For the purposes of this invention, the expression, “β-actin” refers to sequences (DNA and/or RNA), as well as respective proteins, comprising at least 50% homology with the sequences (DNA and/or RNA), as well as respective proteins of the β-actin gene.

Ideally, the β-actin sequence is described in the GenBank under the access number, NM_{—}001101.

Cells comprising at least of gene of the sirtuin family

For the purposes of this invention, adequate cells are cells of vertebrate animals. Preferably, the vertebrate should be chosen from the group comprising fish, birds, mammals and combinations of the same.

Ideally, the vertebrate should be chosen from the group comprising Danio rerio, Gallus gallus, Mus musculus, Homo sapiens and combinations of the same.

The cell is chosen from different tissues in the animal. Examples of organs/tissues include, but are not limited to, the brain, kidney, sexual organs (male and female), spleen, gills, liver, muscle, heart and combinations of the same.

The zebrafish (Danio rerio) possess numerous advantageous characteristics that firstly make it the principal vertebrate model for biology studies in development and genetics and, subsequently, expansion to other areas of biological sciences. The zebrafish combines the characteristic of being a vertebrate with the study size of an invertebrate organism. The knowledge accumulated from the “Zebrafish Genome Project” together with the capability to quickly absorb chemical substances directly from water have the effect that it is increasingly used as well as a model species for studies in toxicology, pharmacology and human diseases.

The identification of genes related to sirtuins in zebrafish genomes and the determination of expression patterns in different tissues constitute a very powerful tool for screening modulating molecules for the activities of these proteins.

Method for Determining Modulating Agents

The method for determining modulating agents of sirtuin gene expression is comprised of the following stages:
a) contacting cells comprising at least on gene of the sirtuin family and the β-actin gene, with a modulating agent;
b) determining the relative abundance of mRNA of the sirtuin-family gene;
c) determining the relative abundance of the mRNA of the β-actin gene; and
d) determining the mRNA SIRTImRNA β-actin ratio.

Ideally, one determines the relative abundance by way of mRNA amplification tests in both genes. Said tests are common knowledge to experts in the subject and do not need to be described here in greater detail.

In the event that the compound possesses a mRNA SIRTImRNA β-actin ratio that is greater than 1, the compound is classified as a positive modulator (amplifier) of sirtuin gene expression.

In the event that the compound possesses a mRNA SIRTImRNA β-actin ratio that is less than 1, the compound is classified as a negative modulator (inhibitor) of sirtuin gene expression.

Pharmaceutical Composition

For the purposes of this invention, pharmaceutical compositions include all compositions that contain an active principle, with prophylactic, palliative and/or curative purposes, which acts so as to maintain and/or restore homeostasis. It can be administered topically, parenterally, enterally and/or intrathecally.

The expression, “pharmaceutically acceptable”, is employed here when referring to compounds, materials, compositions and/or dosage methods which, within the medical field, are appropriate for use in contact with human and animal tissue without excessive toxicity, irritation, allergic response or other problems or complications, it having a reasonable benefit/risk relationship.

The composition in the invention herein can be administered by way of an oral dosage, in the form of tablets, capsules (each one includes sustained liberation or formulations with liberation time), pills, powders, granules, elixirs, dyes, suspensions, syrups and emulsions.

The pharmaceutical composition in this invention is comprised of:
a) a modulating agent for sirtuin expression; and
b) a vehicle which is acceptable pharmaceutically,
   in which the mRNA SIRTImRNA β-actin ratio of the cell without the presence of the modulating agent is different from the mRNA SIRTImRNA β-actin ratio of the cell without the presence of the modulating agent.

### Example

Eight genes related to sirtuins were identified in the zebrafish genome SIRT1-7 and a SIRT3 paralogue, named S1RT3.2 (Table 1) utilising the sequences deduced from Homo sapiens amino acids (Q96EB6, Q8IXJ6, Q9NTG7, Q9Y6E7, Q9NXA8, Q8N6T7, Q9NRC8) Mus musculus (BAD38898, BAD38897, Xp_{—}420920, XP_{—}415273, XP_{—}418925, NP_{—}001034409, NP_{—}001026277) and Gallus gallus (NP_{—}062786, NP_{—}071877, CAJ18608, Q8R216, NP_{—}849179, NP_{—}853617, AAP83960). The identity of each one of the eight sequences of zebrafish was confirmed by way of phylogenetic analysis (FIG. 1)

TABLE 1

Sequences Summary: Access number, primers sequence and PCR
amplification conditions.

PCR Conditions

| | | | Tm | |
|---|---|---|---|---|
| Sirtuin | GenBank ID | Primers (5′-3′) | (° C.) | Ciclos |
| SIRT 1 | XP_001334440 | F-CAGCTCTGCTACAATTCATCGCGTC | 62 | 30 |
| | | R-AATCTCTGTAGAGTCCAGCGCGTGTG | | |
| SIRT 2 | AAH67165.1 | F-TCTCTGAAGAAATTCCTAAGTGCGATTCC | 61 | 30 |
| | | R-TTATCTGAATCAAAATCCATTCCGCCTC | | |
| SIRT 3 | NP_001073643 | F-CATTAAATGTGGTGGAACAAGAGGCCTG | 61 | 30 |
| | | R-AGTTCCTCTCCTTTGTAATCCCTCCGAC | | |
| SIRT 3.2 | NP_001038173.1 | F-CGGCAGGCTGATGAAGCTTGGTCG | 63 | 30 |
| | | R-TAGCTTGCTTGGCTTCCTCTGCAGG | | |
| SIRT 4 | AAH83418.1 | F-TGTGGTGAACTGACTCCTCGTGCTGAGC | 63 | 30 |
| | | R-CGGAAGTTTTCTTTCACTAGCAGCGAGG | | |
| SIRT 5 | AAH75987.1 | F-CCACGGTAGTCTGTTTAAAACCCGCTG | 61 | 30 |
| | | R-AGTGATATTTGAAGCGTTGGGTAGCAGG | | |
| SIRT 6 | NP_001002071 | F-GGACTGGGAGGACTCTCTGCCCGAC | 68 | 30 |
| | | R-GCCCGGCCCACTCCGGAACG | | |
| SIRT 7 | AA155852.1 | F-GCATTTTGGAGAACGTGGCACTTTGG | 61 | 45 |
| | | R-GTTTAGCCATGCTGAAGATGGGGTCC | | |

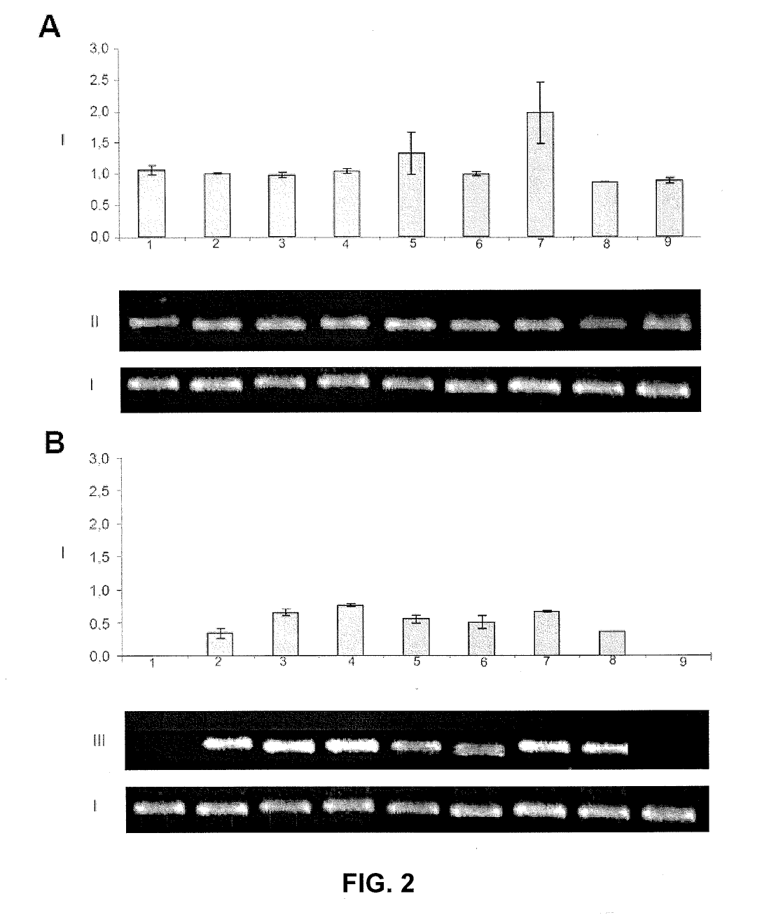

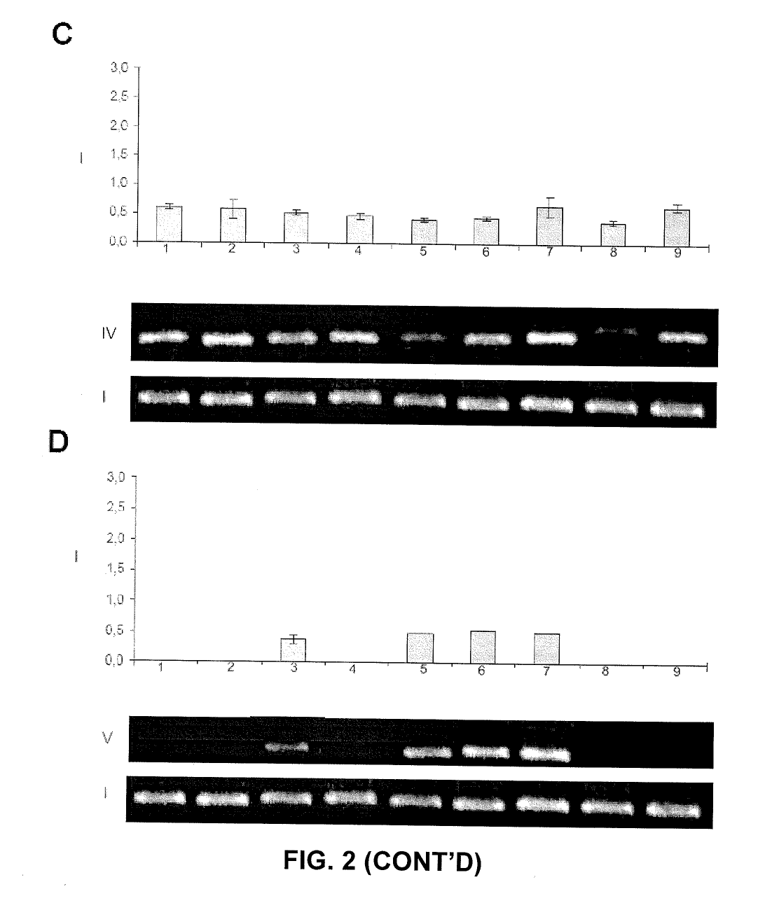

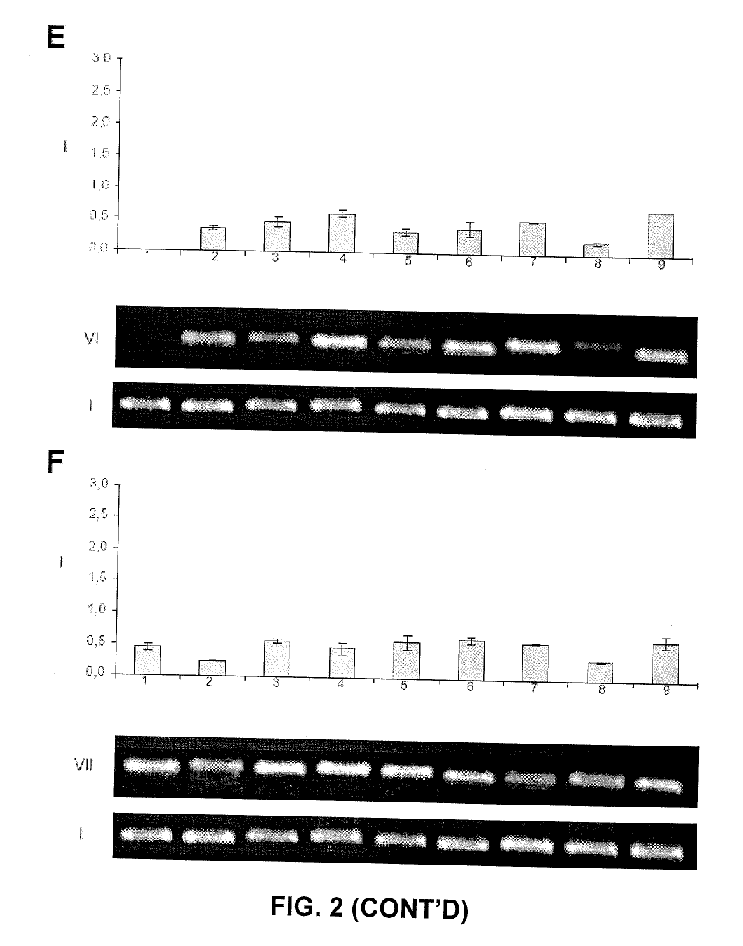

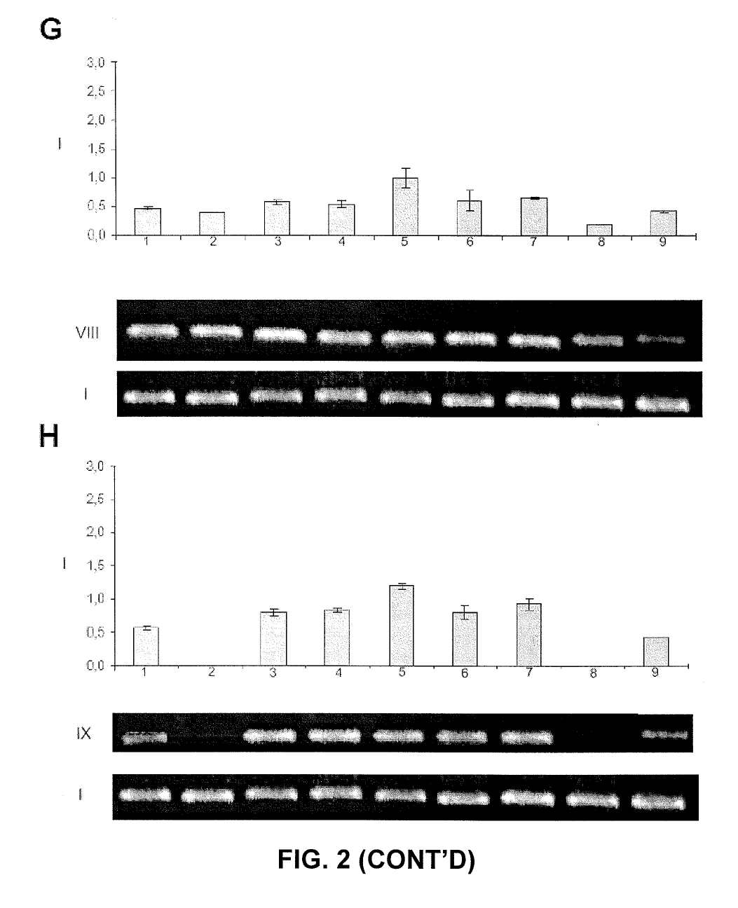

The mapping of the pattern and levels of expression in each one of the eight genes related to zebrafish sirtuin was determined in eight tissues (spleen, gills, brain, heart, liver, female sexual organs and male sexual organs, skeletal muscle and kidneys), by way of the analysis of semi-quantitative RT-PCR comparing the relative abundance of mRNA of each one of the genes related to sirtuins with the mRNA of the gene codifying for β-actin (mean ±E.P.) (FIG. 2 and Table 2).

TABLE 2
Gene expression pattern: relative expression of mRNA in genes related
to sirtuin in different zebrafish tissues.

| | Optical Density (O.D.) for SIRTIβ-actin mRNA ratio (mean ± S.E) | | | | | | | |
|---|---|---|---|---|---|---|---|---|
| Tissues | SIRT1 | SIRT2 | SIRT3 | SIRT3.2 | SIRT4 | SIRT5 | SIRT6 | SIRT7 |
| Spleen | 1.07 ± 0.07 | * | 0.61 ± 0.05 | * | * | 0.44 ± 0.05 | 0.47 ± 0.03^{g} | 0.56 ± 0.04^{d,g} |
| Gills | 1.01 ± 0.01 | 0.34 ± 0.00^{a,d,e} | 0.57 ± 0.16 | * | 0.36 ± 0.10^{f} | 0.24 ± 0.01^{c,f} | 0.38 ± 0.01^{g} | * |
| Brain | 0.98 ± 0.05 | 0.65 ± 0.08^{b} | 0.52 ± 0.04 | 0.37 ± 0.03 | 0.46 ± 0.03 | 0.55 ± 0.03 | 0.56 ± 0.04^{g,h} | 0.79 ± 0.10^{g} |
| Heart | 1.05 ± 0.04 | 0.77 ± 0.05^{b,h} | 0.46 ± 0.05 | * | 0.61 ± 0.07^{h} | 0.45 ± 0.09 | 0.53 ± 0.06^{g,h} | 0.83 ± 0.05 |
| Liver | 1.33 ± 0.34 | 0.56 ± 0.02 | 0.41 ± 0.04 | 0.49 ± 0.09 | 0.33 ± 0.06^{f} | 0.56 ± 0.12 | 0.98 ± 0.17^{a,b,c.d,e,f,h} | 1.19 ± 0.04^{a,c,f,i} |
| Female | 1.00 ± 0.04 | 0.51 ± 0.06 | 0.43 ± 0.03 | 0.54 ± 0.07 | 0.39 ± 0.06 | 0.61 ± 0.05^{b} | 0.59 ± 0.17^{g,h} | 0.79 ± 0.05^{g} |
| Sexual Organ | | | | | | | | |
| Male | 1.97 ± 0.49^{h,i} | 0.68 ± 0.09^{b} | 0.64 ± 0.17 | 0.51 ± 0.08 | 0.52 ± 0.12^{h} | 0.56 ± 0.01 | 0.65 ± 0.03^{g,h} | 0.92 ± 0.11^{f,i} |
| Sexual Organ | | | | | | | | |
| Skeletal | 0.87 ± 0.01^{g} | 0.37 ± 0.01^{e} | 0.38 ± 0.05 | * | 0.19 ± 0.01^{e,d,f} | 0.29 ± 0.02^{f} | 0.19 ± 0.00^{a,c.d,e,f} | * |
| Muscle | | | | | | | | |
| Kidney | 0.89 ± 0.04^{g} | * | 0.64 ± 0.06 | * | 0.69 ± 0.03^{b,g,m} | 0.62 ± 0.09^{b,h} | 0.41 ± 0.02^{g} | 0.45 ± 0.09^{d,g} |

The results were analysed by ANOVA followed by the post-hoc Tukey HSD test, considering P ≦ 0.05 as significant.
The mRNA levels were significantly different from
^{a}spleen,
^{b}gills,
^{c}brain,
^{d}heart,
^{e}liver,
^{f}female sexual organ,
^{g}male masculine organ,
^{h}skeletal muscle,
ⁱkidney.

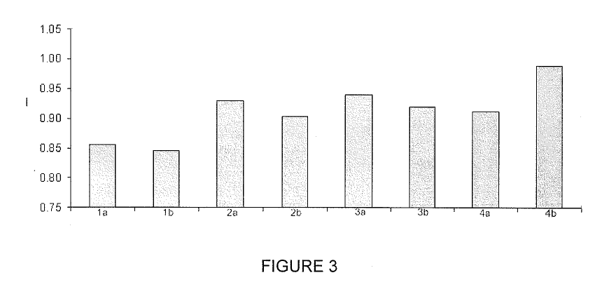

So as to evaluate modulation in this protein family, the effect of resveratrol (3.4′,5-trihydroxy-trans-stilbene), a phytoalexin which is found is some food items, such as egg shell, peanuts and red wine, was evaluated and it promoted a significant increase in SIRT1 gene expression in skeletal muscle and liver in the proposed model (FIG. 3).

1. A method for determining modulating agents of sirtuins comprising the following steps:
a. contacting tissue cell(s) comprising at least of gene of the sirtuin family and the β-actin gene, with a modulating gene;
b. determining the relative abundance of mRNA of the sirtuin-family gene;
c. determining the relative abundance of the mRNA of the β-actin gene; and
d. determining the mRNA SIRTImRNA β-actin ratio.

2. The method for determination, pursuant to claim 1, in which cells are selected from the group consisting of cells of fish, birds, mammals and combinations thereof.

3. The method for determination, pursuant to claim 2, in which the fish cells are cells from Danio rerio.

4. The method for determination, pursuant to claim 2, in which the fish cells are cells from Gallus gallus.

5. The method for determination, pursuant to claim 2, in which the mammal cells are from Mus musculus and/or Homo Sapiens cells.

6. The method for determination, pursuant to claim 2, in which the tissue is selected from the group consisting of brain, kidney, sexual organs (male and female), spleen, gills, liver, muscle, heart and combinations thereof.

7. The method for determination, pursuant to claim 1, in which the sirtuin possesses at least 50% of homology with at least one sequence selected from the group consisting of XP_{—}001334440 (SEQ ID NO: 1), AAH67165 (SEQ ID NO: 2), XP_{—}684225 (SEQ ID NO: 3), XP_{—}690925 (SEQ ID NO: 4), AAH83418 (SEQ ID NO: 5), AAH75987 (SEQ ID NO: 6), NP_{—}001002071 (SEQ ID NO: 7), XP_{—}698800 (SEQ ID NO: 8), Q96EB6 (SEQ ID NO: 9), Q8IXJ6SEQ ID NO: 10), Q9NTG7 (SEQ ID NO: 11), Q9Y6E7 (SEQ ID NO: 12), Q9NXA8 (SEQ ID NO: 13), Q8N6T7 (SEQ ID NO: 14), Q9NRC8 (SEQ ID NO: 15), BAD38898 (SEQ ID NO: 16), BA[[S]]D38897 (SEQ ID NO: 17), XP_{—}420920 (SEQ ID NO: 18), XP_{—}415273 (SEQ ID NO: 19), XP_{—}418925 (SEQ ID NO: 20), NP_{—}001034409 (SEQ ID NO: 21), NP_{—}001026277 (SEQ ID NO: 22), NP_{—}062786 (SEQ ID NO: 23), NP_{—}071877 (SEQ ID NO: 24), CAJ18608 (SEQ ID NO: 25), Q8R216 SEQ ID NO: 26), NP_{—}849179 (SEQ ID NO: 27), NP_{—}853617 (SEQ ID NO: 28), AAP83960 (SEQ ID NO: 29) and combinations thereof.

8. The method for determination, pursuant to claim 1, in which the β-actin possess at least 50% of homology with the NM_{—}001101 (SEQ ID NO: 30) sequence.

9. The method for determination, pursuant to claim 1, in which the relative abundance of mRNA of sirtuins and/or β-actin is determined by PCR.

10. A kit for determining modulating agents of sirtuins comprising:
a. means for determining the relative abundance of mRNA of the sirtuin-family gene; and
b. means for determining the relative abundance of mRNA of the β-actin gene.

11. The kit for determining, in accordance with claim 10, in which the means for determining the relative abundance of mRNA of sirtuins and/or β-actin is comprised of primers capable of aligning themselves with a sequence of nucleotides possessing at least 50% of homology with at least one sequence selected from the group consisting of XP_{—}001334440 (SEQ ID NO: 1), AAH67165 (SEQ ID NO: 2), XP_{—}684225 (SEQ ID NO: 3), XP_{—}690925 (SEQ ID NO: 4), AAH83418 (SEQ ID NO: 5), AAH75987 (SEQ ID NO: 6), NP_{—}001002071SEQ ID NO: 7), XP_{—}698800 (SEQ ID NO: 8), Q96EB6 (SEQ ID NO: 9), Q8IXJ6 (SEQ ID NO: 10), Q9NTG7 (SEQ ID NO: 11), Q9Y6E7 (SEQ ID NO: 12), Q9NXA8 (SEQ ID NO: 13), Q8N6T7 (SEQ ID NO: 14), Q9NRC8 (SEQ ID NO: 15), BAD38898 (SEQ ID NO: 16), BA[[S]]D38897 (SEQ ID NO: 17), XP_{—}420920 (SEQ ID NO: 18), XP_{—}415273 (SEQ ID NO: 19) XP_{—}418925 (SEQ ID NO: 20), NP_{—}001034409 (SEQ ID NO: 21), NP_{—}001026277 (SEQ ID NO: 22), NP_{—}062786 (SEQ ID NO: 23), NP_{—}071877 (SEQ ID NO: 24), CAJ18608 (SEQ ID NO: 25), Q8R216 (SEQ ID NO: 26), NP_{—}849179 (SEQ ID NO: 27), NP_{—}853617 (SEQ ID NO: 28), AAP83960 (SEQ ID NO: 29), NM-001101 (SEQ ID NO: 30) and combinations thereof.

12. A process for modulating sirtuin expression comprising contacting a modulating agent of sirtuin expression with a cell comprised of at least one sirtuin-family gene.

13. The modulation process, pursuant to claim 12, in which the cells are selected from the group consisting of cells of fish, birds, mammals and combinations thereof.

14. The modulation process, pursuant to claim 13, in which the fish cells are Danio rerio cells.

15. The modulation process, pursuant to claim 13, in which the bird cells are Gallus gallus cells.

16. The modulation process, pursuant to claim 13, in which the mammal cells are Mus musculus and/or Homo Sapiens cells.

17. The modulation process, pursuant to claim 13, in which the tissue is selected from the group consisting of brain, kidney, sexual organs (male and female), spleen, gills, liver, muscle, heart and combinations thereof.

18. The modulation process, pursuant to claim 12, in which the sirtuin possesses at least 50% of homology with at least one sequence selected from the group consisting of XP_{—}001334440 (SEQ ID NO: 1), AAH67165 (SEQ ID NO: 2), XP_{—}684225 (SEQ ID NO: 3), XP_{—}690925 (SEQ ID NO: 4), AAH83418 (SEQ ID NO: 5), AAH75987 (SEQ ID NO: 6), NP_{—}001002071 (SEQ ID NO: 7), XP_{—}698800 (SEQ ID NO: 8), Q96EB6 (SEQ ID NO: 9), Q8IXJ6 (SEQ ID NO: 10), Q9NTG7 (SEQ ID NO: 11), Q9Y6E7 (SEQ ID NO: 12), Q9NXA8 (SEQ ID NO: 13), Q8N6T7 (SEQ ID NO: 14), Q9NRC8 (SEQ ID NO: 15), BAD38898 (SEQ ID NO: 16), BA[[S]]D38897 (SEQ ID NO: 17), XP_{—}420920 (SEQ ID NO: 18), XP_{—}415273 (SEQ ID NO: 19) XP_{—}418925 (SEQ ID NO: 20), NP_{—}001034409 (SEQ ID NO: 21), NP_{—}001026277 (SEQ ID NO: 22), NP_{—}062786 (SEQ ID NO: 23), NP_{—}071877 (SEQ ID NO: 24), CAJ18608 (SEQ ID NO: 25), Q8R216 (SEQ ID NO: 26), NP_{—}849179 (SEQ ID NO: 27), NP_{—}853617 (SEQ ID NO: 28), AAP83960SEQ ID NO: 29) and combinations thereof.

19. A pharmaceutical composition comprising:
a. a modulating agent of sirtuin expression; and
b. an acceptable pharmaceutical vehicle,
in which the mRNA SIRTImRNA β-actin ratio of the cell without the presence of the modulating agent is different from the mRNA SIRTImRNA β-actin ratio of the cell without the presence of the modulating agent.

20. The pharmaceutical composition, pursuant to claim 19, in which the modulating agent is resveratrol.

21. A modulating agent of sirtuin expression utilized in the manufacturing of medication which is useful for the treatment of neurodegenerative diseases.
